(19) United States
(12) Patent Application Publication
(10) Pub. No.: US 2023/0210762 A1
(43) Pub. Date: Jul. 6, 2023

(54) TRANSDERMAL DRUG DELIVERY DEVICES HAVING PSILOCYBIN, LYSERGIC ACID DIETHYLAMIDE OR 3,4-METHYLENEDIOXYMETHAMPHETAMINE COATED MICROPROTRUSIONS

(71) Applicant: Emergex USA Corporation, Doylestown, PA (US)

(72) Inventors: Mahmoud AMERI, Fremont, CA (US); Hayley LEWIS, Redwood City, CA (US)

(73) Assignee: Emergex USA Corporation, Doylestown, PA (US)

(21) Appl. No.: 17/997,094

(22) PCT Filed: May 3, 2021
(86) PCT No.: PCT/US21/30437

§ 371 Date: Oct. 25, 2022

Related U.S. Application Data
(60) Provisional application No. 63/018,759, filed on May 1, 2020.

(51) Int. Cl. A61K 9/00; A61K 31/675; A61K 31/48; A61K 31/36; A61K 47/12; A61M 37/00; A61P 25/24

(52) U.S. Cl. CPC A61K 9/0021; A61K 31/675; A61K 31/48; A61K 31/36; A61K 47/12; A61M 37/0015; A61P 25/24; A61M 2037/0061; A61M 2037/0023; A61M 2037/0046

(57) ABSTRACT

Disclosed herein are compositions, devices and methods employing therapeutic concentrations of psilocybin, LSD or MDMA for the treatment of certain health conditions, including depression, anxiety, post-traumatic stress disorder, migraine and cluster headache. Also described are methods and apparatuses to deliver psilocybin, LSD or MDMA by intracutaneous administration via microneedle administration.

### CROSS REFERENCE TO RELATED APPLICATION

This application claims benefit of U.S. Provisional Patent Application No. 63/018,759 filed on May 1, 2020; which is incorporated herein by reference in its entirety to the full extent permitted by law.

### FIELD

The present invention relates to the field of transdermal or intracutaneous delivery of pharmaceutical agents, and more particularly to the delivery of psilocybin, lysergic acid diethylamide (“LSD”) or 3,4-methylenedioxymethamphetamine (“MDMA”).

### BACKGROUND

Psilocybin (3-[2-(demethylamino)ethyl-1H-indol-4-yl] dihydrogen phosphate) is a natural product produced by numerous species of Psilocybe mushrooms. Psilocybin is a tryptamine derivative, and in humans the phosphate group is rapidly enzymatically cleaved in the body to produce psilocin, an agonist at a variety of serotonin receptors, the most important of which in this setting is the 5-HT2A receptor (Carhart-Harris et al., 2014; Nichols, 2004). Psilocybin has a chemical formula of C₁₂H₁₇N₂O₄P, and molar mass of 284.252 g·mol⁻¹. Psilocybin is soluble in water. The chemical structure of psilocybin is:

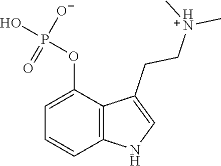

Psilocybin is metabolized mostly in the liver. As psilocybin is converted to psilocin in the liver, it undergoes a first pass effect, whereby its concentration is greatly reduced before it reaches the systemic circulation. The active psilocin is then either glucuronated and excreted in the urine or is further converted to various psilocin metabolites. For example, psilocin is broken down by the enzyme monoamine oxidase to produce several metabolites that can circulate in the blood plasma, including 4-hydroxyindole-3-acetaldehyde, 4-hydroxytryptophol, and 4-hydroxyindole-3-acetic acid. (Passie T., et al., (2002) “The pharmacology of psilocybin,” Addiction Biology, 7(4): 357-64). The psilocin that is not broken down by enzymes instead forms a glucuronide, through linkage with glucuronic acid, that can then be excreted in the urine. (Grieshaber A F, et al., (2001) “The detection of psilocin in human urine,” Journal of Forensic Sciences, 46(3):627-30).

When administered orally, psilocybin as a half-life of 163±64 minutes, compared to when administered intravenously its half-life is 74.1±19.6 minutes. Psilocybin ingested orally has about a 50% bioavailability and psilocin is detectable in plasma within 20 minutes of administration of the parent compound (Brown et al., 2017; Hasler, Bourquin, Brenneisen, Bar, & Vollenweider, 1997). Based on studies in animals, about 50% of ingested psilocybin is absorbed through the stomach and intestine, and within 24 hours about 65% of the absorbed psilocybin is excreted into the urine, and a further 15-20% is excreted in the bile and feces. Although most of the drug is eliminated in this manner within 8 hours, psilocybin has been detected in urine 7 days after ingestion. (Matsushima Y., et al., (2009) “Historical overview of psychoactive mushrooms”, Inflammation and Regeneration, 29 (1): 47-58).

Psilocybin readily induces profound changes in sensory perception, emotion, thought, and sense of self, characterized by marked alterations in all mental functions, including perception, mood, volition, cognition and self-experience (Geyer & Vollenweider, 2008; Studerus, Kometer, Hasler & Vollenweider, 2011). These profound changes are often referred to as mystical-type experiences. Measures of mystical-type experiences occurring during psilocybin treatment have been repeatedly observed to predict later effects on behavior and emotions, including reductions in depressive and anxiety symptoms (Griffiths et al., 2016; MacLean, Johnson, & Griffiths, 2011; Ross et al., 2016).

Non-clinical studies demonstrate that similar to humans, when psilocybin is administered orally to rats it is rapidly dephosphorylated to psilocin in the intestinal mucosa by alkaline phosphatase and a nonspecific esterase, with approximately 50% of the total volume of psilocin absorbed from the digestive tract (Kalberer et al., 1962). Maximum plasma levels are achieved after approximately 90 minutes (Chen et al., 2011). When administered systemically (i.e., bypassing the gut), initial psilocybin metabolism is performed by tissue phosphatases, with in vitro studies indicating the kidneys as being among the most active metabolic organs (Horita & Weber, 1961).

Recent evidence suggests that psychedelic agonists have distinct biological effects not found in non-psychedelic 5HT2A agonists. Psychedelic, but not non-psychedelic, 5HT2A agonists have been shown via receptor-receptor interactions to enhance signaling through the dopamine D2 receptor in ventral striatum, which is of significant interest given that increased dopamine activity in this area correlates with euphoria in response to psilocybin (Vollenweider, Vontobel, Hell, & Leenders, 1999), and given that abnormalities in the D2 receptor have been reported in the same brain area in patients with major depression (Pei et al., 2010). Recent studies indicate that psychedelic and non-psychedelic 5HT2A agonists also differentially regulate intracellular signaling pathways in pyramidal neurons, with resultant differences in the expression of downstream signaling pathways, such as beta-arrestin 2 and early growth response protein 1 (EGR1) (Gonzalez-Maeso et al., 2007; Schmid, Raehal, & Bohn, 2008). Although 5HT2A agonism is widely recognized as the primary action of classic psychedelic agents, psilocybin has lesser affinity for a wide range of other pre- and post-synaptic serotonin and dopamine receptors, as well as the serotonin reuptake transporter (Tyls, Palenicek, & Horacek, 2014). Psilocybin activates 5HT1A receptors, which may contribute to antidepressant/anti-anxiety effects.

The molecule 3,4-methylenedioxymethamphetamine (MDMA) is a chiral molecule possessing two enantiomers, S(+)-MDMA and R(−)-MDMA, with S(+)-MDMA being more potent than R(−)-MDMA (Shulgin, A. T., The background and chemistry of MDMA. J Psychoactive Drugs, 1986. 18(4): p. 291-304: Lyon, R.A., R.A. Glennon, and M. Titeler, 3,4-Methylenedioxymethamphetamine (MDMA): stereoselective interactions at brain 5-HT1 and 5-HT2 receptors. Psychopharmacology (Berl), 1986. 88(4): p. 525-6). Research in humans to date and the majority of nonclinical studies have used racemic MDMA, or an admixture containing equal amounts of both enantiomers. Studies of drug discrimination in rodents and studies of self-administered MDMA enantiomers in primates suggest that MDMA enantiomers may produce different physiological and rewarding effects, and there may be some synergy between the two when administered as a racemate (Fantegrossi, W. E., et al., Discriminative stimulus effects of 3,4-methylenedioxymethamphetamine and its enantiomers in mice: pharmacokinetic considerations. J Pharmacol Exp Ther, 2009. 329(3): p. 1006-15; Yarosh, H. L., et al., MDMA-like behavioral effects of N-substituted piperazines in the mouse. Pharmacol Biochem Behav, 2007. 88(1): p. 18-27]Murnane, K. S., et al., Endocrine and neurochemical effects of 3,4-methylenedioxymethamphetamine and its stereoisomers in rhesus monkeys. J Pharmacol Exp Ther, 2010. 334(2): p. 642-50; Fantegrossi, W. E., et al., 3,4-Methylenedioxymethamphetamine (MDMA, “ecstasy”) and its stereoisomers as reinforcers in rhesus monkeys: serotonergic involvement. Psychopharmacology (Berl), 2002. 161(4): p. 356-64; Fantegrossi, W. E., et al., Role of dopamine transporters in the behavioral effects of 3,4-methylenedioxymethamphetamine (MDMA) in nonhuman primates. Psychopharmacology (Berl), 2009; McClung, J., W. Fantegrossi, and L. L. Howell, Reinstatement of extinguished amphetamine self-administration by 3,4-methylenedioxymethamphetamine (MDMA) and its enantiomers in rhesus monkeys. Psychopharmacology (Berl), 2010. 210(1): p. 75-83; Murnane, K. S., et al., The neuropharmacology of prolactin secretion elicited by 3,4- methylenedioxymethamphetamine (“ecstasy”): a concurrent microdialysis and plasma analysis study. Horm Behav, 2012. 61(2): p. 181-90.).

LSD possesses a complex pharmacological profile that includes direct activation of serotonin, dopamine and norepinephrine receptors. In addition, one of its chief sites of action is that of compound-specific (“allosteric”) alterations in secondary messengers associated with 5HT2A and 5HT2C receptor activation and changes in gene expression. The hallucinogenic effects of LSD are likely due to agonism at 5HT2A and 5HT2C receptors (Aghajanian and Marek 1999; Nichols 2004), with at least one drug discrimination study in rats finding that a 5HT2A receptor antagonist (ritanserin) was more successful than a 5HT2C antagonist (SB 46349B) at eliminating LSD stimulus cues (Appel, West et al. 2004). However, LSD is also an agonist at the majority of known serotonin receptors, including 5HT1A, 5HT1B, 5HT1D, 5HT5A, 5HT6 and 5HT7 receptors (Boess and Martin 1994; Eglen, Jasper et al. 1997; Hirst, Abrahamsen et al. 2003; Nichols and Sanders-Bush 2002). The only serotonin receptor for which LSD fails to show significant affinity is the 5HT3 receptor, the only serotonin receptor that is a ligand-gated ion channel rather than a G-protein coupled receptor.

LSD also has affinity for dopamine D1 and D2 receptors (Creese et al. 1975; Nichols et al. 2002). Drug discrimination studies in rodents suggest that dopamine receptors may play a role in producing effects appearing after and in addition to changes associated with 5HT2A activation (Marona-Lewicka and Nichols 2007; Marona-Lewicka et al. 2005), and behavioral observations suggest that LSD may produce some effects through the dopamine system (Burt, Creese et al. 1976; Chiu and Mishra 1980; Watts, Lawler et al. 1995). There is some evidence that LSD has affinity for alpha adrenergic receptors (Marona-Lewicka and Nichols 1995; U'Prichard et al. 1977). Clonidine potentiated the LSD stimulus in rats trained to recognize LSD, an effect that suggests at least indirect action on these receptors (Marona-Lewicka and Nichols 1995). By contrast, LSD appears to have little to no affinity for histamine receptors (Green, Weinstein et al. 1978; Nichols, Frescas et al. 2002), and the only evidence of action at acetylcholine sites is indirect. An in vitro study also suggests that LSD is an agonist at trace amine receptors (TAR) (Bunzow, Sonders et al. 2001). The functional significance of activity at trace amine receptors remains unclear, given that stimulants and entactogens (MDMA-like drugs) also activate these receptors (Bunzow, Sonders et al. 2001; Miller, Verrico et al. 2005).

Clinical studies involving psilocybin, MDMA and LSD have been completed, are ongoing, or are being contemplated, in order to evaluate their potential as an effective treatment for a variety of health conditions including depression, including major depressive disorder, anxiety, grief, post-traumatic stress disorder, Alzheimer Disease, mild cognitive impairment, obsessive-compulsive disorder, anorexia nervosa, migraine headache, cluster headache, post-traumatic headache, alcohol dependence, nicotine dependence, opioid use disorder, cocaine-related disorders, stage IV melanoma, cancer, Parkinson Disease, psychosis, adolescent behavior, adolescent development, and altered waking states of consciousness.

Synthetic versions of psilocybin for oral administration have previously been developed that are suitable for use in pharmaceutical formulations. U.S. Pat. No. 10,519,175 describes orally administering a dosage form that comprises crystalline psilocybin, or different polymorphic forms of psilocybin, including isostructural variants, such as Polymorph A, Polymorph A′, Polymorph B, and other forms such as hydrates.

The currently available methods of administering psilocybin, LSD and MDMA involve oral administration, which has several disadvantages. For example, some patients with the targeted health conditions discussed above fail to respond consistently to oral administration, and oral treatments may be ineffectual and/or unpleasant for patients who are suffering from the nausea, vomiting, or gastrointestinal issues that can be associated with the targeted health conditions. Oral products are also characterized by delayed absorption and relatively slow onset of action causing insufficient relief, especially early in the episode. This delay is a critical failure in health conditions such as migraine and cluster headache, where cluster headache episodes in particular often last for 30 minutes or less, rendering oral administration treatments ineffective.

In addition, as noted above, when psilocybin is administered orally, only about 50% of the administered psilocybin is actually absorbed by the stomach and intestine, requiring a larger dosage to achieve therapeutically effective concentrations. The psilocybin that is absorbed undergoes first-pass metabolism in the liver, which produces several metabolites that can circulate in the blood. Not only does oral administration of psilocybin require large doses and produce several undesirable metabolites, but the psilocybin also stays in the body longer than when administered intravenously, as the half-life of psilocybin administered orally is 163±64 minutes.

Although not currently applied for the therapeutic use of psilocybin, LSD and MDMA, several traditional non-oral routes of drug administration also have disadvantages that make their use unfavorable. For example, transdermal iontophoresis, patches, and liquid injectors have the disadvantages of skin irritation and scarring, pain and the inability to deliver a therapeutically effective dose. Subcutaneous injection likewise has negatives associated with its use, such as it is difficult to prepare, needle phobia, sharps disposal, accidental pricking, cutting, and cross contamination that is related to delivery with a needle.

Therefore, advantages could be achieved by a therapeutic alternative to oral administration of psilocybin, LSD and MDMA that: (a) has an onset of action faster than oral administration, but without issues related to needles; (b) avoids the oral route that may limit absorption caused by the gastric effects of some of the intended health conditions, such as gastric stasis, nausea, and vomiting associated with migraines; (c) mitigates the potential for food interactions, avoids first-pass metabolism and reduces the potential for drug interactions; (d) is preferred by patients (rapid onset but not injected or with unpleasant taste/smell); (e) has lower absorption that reduces side effects; (f) can be conveniently carried by the patient for use at the first sign of an episode associated with the targeted health conditions; and (g) can be quickly, conveniently, and safely self-administered.

Thus, there is a need in the art for a route of administration that can effectively deliver doses of psilocybin, LSD or MDMA without the side effects of orally administration, or the side effects associated with other non-oral administration routes. The present disclosure meets these challenges and needs, among others. For instance, Applicant believes that transdermal delivery of psilocybin, LSD or MDMA can rapidly deliver therapeutically effective amounts of psilocybin, LSD or MDMA with plasma concentrations in the range of or higher than those seen following oral administration, despite the difficulty of the skin's impermeable nature.

There is furthermore a need for a route of psilocybin, LSD or MDMA administration that has improved efficacy due to an absorption rate comparable to injection and is portable and easy to prepare while avoiding the issues of needle phobia, sharps disposal, and accidental pricking, cutting, and cross contamination that are related to delivery with a needle.

Further, there is a need in the art for an effective method of psilocybin, LSD or MDMA administration through transdermal delivery in which the patch can be accurately and evenly coated, without causing issues of residual drug on the array or issues of manufacturing inconsistencies, such as uneven formulation coating on a patch or difficulty with formulation sticking to the patch. Many attempts have been made to use transdermal microneedle patches for effective drug delivery; however, achieving rapid release of drug from and rapid treatment with microneedle systems, optimizing and developing effective microneedle shapes and sizes, while also containing a sufficient dosage of drug has proved elusive.

### SUMMARY

The present disclosure relates to compositions, devices, methods of treatment, kits and methods of manufacture of pharmaceutical products useful in the treatment of a variety of health conditions, including migraines, cluster headaches, depression, major depressive disorder, anxiety, grief, post-traumatic stress disorder (PTSD), Alzheimer Disease, mild cognitive impairment, obsessive-compulsive disorder, anorexia nervosa, cluster headache, post-traumatic headache, alcohol dependence, nicotine dependence, opioid use disorder, cocaine-related disorders, stage IV melanoma, cancer, Parkinson Disease, psychosis, adolescent behavior, adolescent development, and altered waking states of consciousness. More specifically, the disclosure is directed to administration of psilocybin, LSD or MDMA as the active pharmaceutical ingredient to a subject in need thereof, i.e., to transdermally or intracutaneously, or otherwise through the skin, administering a therapeutically effective dose of psilocybin, LSD or MDMA that is dose sparing as compared to an oral dose, in a format that is easy to use and portable for rapid administration.

In particular, the present disclosure provides a method to deliver psilocybin, LSD or MDMA intracutaneously via microneedle administration. In one embodiment, the transdermal delivery of psilocybin, LSD or MDMA generally comprises a patch assembly having a microprojection member that includes a plurality of microprojections (or “needles” or “microneedles” or “array”) that are coated with, in fluid contact with a reservoir of, or otherwise comprise the drug. The patch assembly further comprises an adhesive component, and in a preferred embodiment the microprojection member and adhesive component are mounted in a retainer ring. The microprojections are applied to the skin to deliver the drug to the bloodstream, or more particularly, are adapted to penetrate or pierce the stratum corneum at a depth sufficient to provide a therapeutically effective amount to the bloodstream. In one embodiment, the insertion of the drug-coated microneedles into the skin is controlled by a handheld applicator that imparts sufficient impact energy density in less than about 10 milliseconds.

Preferably, the microprojection member includes a biocompatible coating formulation comprising the drug, such as psilocybin, LSD or MDMA, in a dose sufficient to provide therapeutic effect. The coating may further comprise one or more excipients or carriers to facilitate the administration of the drug across the skin. For instance, the biocompatible coating formulation comprises psilocybin, LSD or MDMA and a water-soluble carrier that is first applied to the microprojections in liquid form and then dried to form a solid biocompatible coating.

In a preferred embodiment, psilocybin, LSD or MDMA, excipients, the coating and drying process lead to a drug coating that is non-crystalline (amorphous) with a surprisingly rapid dissolution rate. In this embodiment, the coating, upon its application to the skin via the microneedles, dissolves at a rate sufficient for rapid uptake of the drug into the epidermis and bloodstream. In one embodiment, such rate is less than 20 minutes, or less than 15 minutes, or less than 10 minutes, or less than 5 minutes, or less than 2.5 minutes, or less than 1 minute. This rate leads to rapid migraine and cluster headache relief, as well as relief for depression, anxiety, or PTSD.

Preferably, this rapid uptake leads to greater than about 10% of patients being pain or symptom free in 1 hour after administration, more preferably greater than about 20% of patients, most preferably about 25% of patients or more are pain or symptom free. In another embodiment, this rapid uptake leads to greater than 40% of patients achieving pain or symptom relief in 1 hour after administration, or greater than 50 percent of patients, or about 65% of patients or more achieve pain or symptom relief 1 hour after administration. Preferably, the drug coating is shelf-stable, and remains amorphous for 1 year, more preferably 2 years, following gamma or e-beam irradiation.

Such intracutaneous delivery system may be in the form of a device that is adapted for easy use directly by the patient. For example, the system may be a drug-device combination product comprising: (a) a disposable microprotrusion member with titanium microneedles that are coated with a drug product formulation and dried, the microprotrusion member being centered on an adhesive backing thus forming a patch, and (b) a reusable handheld applicator that ensures the patch is applied to the skin with a defined application energy sufficient to press the microneedles into the stratum corneum thereby resulting in drug absorption. In one embodiment, the delivery system comprises a patch comprising about 0.2 mg to about 10 mg psilocybin, LSD or MDMA, or about 1 mg to about 4 mg, or about 1 mg, or about 1.9 mg, or about 2 mg, or about 3 mg, or about 3.8 mg, or about 4 mg, or about 5 mg, or about 6 mg, or about 7 mg, or about 8 mg, or about 9 mg psilocybin, LSD or MDMA. In one embodiment, the delivery system is designed to deliver about 0.2 mg to about 10 mg psilocybin, LSD or MDMA intracutaneously, or about 1 mg to about 4 mg, or about 1 mg, or about 1.9 mg, or about 2 mg, or about 3 mg, or about 3.8 mg, or about 4 mg, or about 5 mg, or about 6 mg, or about 7 mg, or about 8 mg, or about 9 mg, or more than about 1 mg, or more than about 1.9 mg, or more than about 2 mg, or more than about 3 mg, or more than about 3.8 mg, or more than about 4 mg, or more than about 5 mg, or more than about 6 mg, or more than about 7 mg, or more than about 8 mg or more than about 9 mg psilocybin, LSD or MDMA. In other embodiments, the dose is up to about 2 mg per 3 cm².

In another embodiment, the present disclosure relates to a method for transdermally or intracutaneously administering psilocybin, LSD or MDMA to a patient in need thereof, comprising the steps of: (a) providing a transdermal patch adapted to intracutaneously deliver psilocybin, LSD or MDMA, comprising a microprojection member having a plurality of microprojections that are adapted to penetrate or pierce the stratum corneum of the patient, wherein the microprojections comprise a biocompatible coating partially or fully disposed on the microprojections, the coating comprising a therapeutically effective amount of the psilocybin, LSD or MDMA; and (b) applying the microprojection member of the device to the skin of the patient, whereby the plurality of microprojections penetrate or pierce the stratum corneum and deliver the psilocybin, LSD or MDMA to the patient's bloodstream. In one embodiment, the psilocybin, LSD or MDMA is coated on the microprojections in a total amount of approximately 0.2 to 10 mg of which approximately 50%, or 60%, or 65%, or 75%, or 80%, or 85%, or 90%, or 95%, or 100% of such dose reaches the bloodstream of the patient after administration, preferably wherein more than approximately 50%, or 60%, or 65%, or 75%, or 80%, or 85%, or 90%, or 95% of such dose reaches the bloodstream of the patient after administration.

The present disclosure encompasses a method for treatment or alleviation of depression, anxiety, PTSD, migraine or cluster headache in a human patient in need thereof, comprising the transdermal or intracutaneous administration of a therapeutically effective amount of psilocybin, LSD or MDMA that produces a therapeutic concentration of psilocin (active metabolite of psilocybin), LSD or MDMA in the bloodstream faster than therapeutically effective doses administered orally, intranasally, sublingually, or iontophoretically. In one aspect, the method for treatment or alleviation of depression, anxiety, PTSD, migraine or cluster headache in a patient results in a plasma Tₘₐₓ as quick as about 2 minutes and not later than about 30-40 minutes in most subjects. In another aspect, the method results in a maximum plasma concentration (Cₘₐₓ) of psilocin, LSD or MDMA of less than 13 ng/mL.

Additional embodiments of the present devices, compositions, methods and the like will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description and claims, particularly when considered in conjunction with the accompanying examples and drawings.

### DETAILED DESCRIPTION

Various aspects and embodiments will be described herein. These aspects and embodiments may, however, be embodied in many different forms and should not be construed as limiting; rather, these embodiments are provided so the disclosure will be as thorough and complete so as to inform a person of skill how to make and use the compositions, devices, methods of treatment, kits and methods of manufacture of pharmaceutical products described herein. The terminology used herein is for the purpose of describing the compositions, devices, methods of treatment, kits and methods of manufacture described herein, and is not intended to be limiting unless expressly stated, because the scope of the invention will be limited only by claims accompanying this application and claims accompanying continuation and divisional applications derived therefrom. All books, publications, patents, and patent applications cited herein are hereby incorporated by reference in their entirety.

As can be appreciated from the foregoing and following description, each and every feature described herein, and each and every combination of two or more of such features, is included within the scope of the present disclosure provided that the features included in such a combination are not mutually inconsistent. For example, any embodiment whose use is consistent with any other embodiment is contemplated and thus included in this description. Other aspects and embodiments are set forth in the following description and claims, and also when considered in conjunction with the accompanying examples and drawings.

As used in this specification and the appended claims, the singular forms “a,” “an,” and “the” include plural references unless the context clearly dictates otherwise. For example, a reference to “a method” includes one or more methods, and/or steps of the type described herein and/or which will become apparent to those persons skilled in the art upon reading this disclosure.

### A. DEFINITIONS

Unless defined otherwise, all terms and phrases used herein include the meanings that the terms and phrases have attained in the art, unless the contrary is clearly indicated or clearly apparent from the context in which the term or phrase is used. Any methods and materials similar or equivalent to those described herein can be used in the practice or testing of the present invention, including the particular methods and materials described herein.

Unless otherwise stated, the use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon factors known to those skilled in the art. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden or narrow a particular numerical value or range. As a general matter, “about” or “approximately” broaden the numerical value. The disclosure of ranges is intended as a continuous range including every value between the minimum and maximum values plus the broadening of the range afforded by the use of the term “about” or “approximately.” Consequently, recitation of ranges of values herein are intended to serve as a shorthand method of referring individually to each separate value falling within the range, and each separate value is incorporated into the specification as if it were individually recited herein.

The term “amorphous” means a non-crystalline solid, i.e., a solid that lacks the long-range order that is characteristic of a crystal.

The term “area under the curve” or “AUC” means the area under the curve (mathematically known as definite integral) in a plot of concentration of drug in blood plasma against time. Typically, the area is computed starting at the time the drug is administered and ending when the concentration in plasma is negligible. In practice, the drug concentration is measured at certain discrete points in time and the trapezoidal rule is used to estimate AUC.

The term “biocompatible coating,” as used herein, means and includes a coating formed from a “coating formulation” that has sufficient adhesion characteristics and no (or minimal) adverse interactions with the biologically active agent (a/k/a active pharmaceutical ingredient, or therapeutic agent, or drug).

The term “bioequivalent,” as used herein, denotes a scientific basis on which two or more pharmaceutical products, compositions or methods containing same active ingredient are compared with one another. “Bioequivalence” means the absence of a significant difference in the rate and extent to which the active agent in pharmaceutical equivalents or pharmaceutical alternatives becomes available at the site of action when administered in an appropriately designed study. Bioequivalence can be determined by an in vivo study comparing a pharmacokinetic parameter for the two compositions. Parameters often used in bioequivalence studies are Tₘₐₓ, Cₘₐₓ, AUC_{0-inf}, AUC₀₋ₜ. In the present context, substantial bioequivalence of two compositions or products is established by 90% confidence intervals (CI) of between 0.80 and 1.25 for AUC and Cₘₐₓ.

The term “cluster headache” as used herein refers to a condition characterized by excruciating headache pain that recurs, usually daily (although bouts may recur up to 8 times per day), for a period of a week or longer. Cluster headaches pain is usually localized on one side of the head (“unilateral”) usually the same side although in some patients the side can vary. The pain usually reaches full intensity in under 10 minutes and lasts for between 15 minutes and 3 hours (usually between 30 and 60 minutes). Because of the rapid onset of symptoms and short duration, treatment via a route by which the drug is rapidly absorbed is required.

The term “coating formulation,” as used herein, means and includes a freely flowing composition or mixture, which is employed to coat a delivery surface, including one or more microprojections and/or arrays thereof

The term “desiccant,” as used herein, means an agent that absorbs water, usually a chemical agent.

The term “electrotransport,” as used herein, refers in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may be attained in various manners.

The term “excipients” refers to inert substances that are commonly used as a diluent, vehicle, preservative, binder, stabilizing agent, etc. for drugs and includes, but is not limited to, proteins (e.g., serum albumin, etc.), amino acids (e.g., aspartic acid, glutamic acid, lysine, arginine, glycine, histidine, leucine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See also Remington's Pharmaceutical Sciences, 21^{st} Ed., LWW Publisher (2005) for additional pharmaceutical excipients.

The term “headache pain scale” as used herein, means a scale used to allow patients to quantify their level of pain. Preferably a scale of 0-3 is used, wherein severe pain has a pain score of 3, moderate pain has a score of 2, mild pain has a score of 1, and no pain (also referred to as “pain freedom”) has a score of 0.

The word “intracutaneous” or “transdermal” as used herein, is a generic term that refers to delivery of an active agent (e.g., a therapeutic agent, such as a drug, pharmaceutical, peptide, polypeptide or protein) through the skin to the local tissue or systemic circulatory system without substantial cutting or penetration of the skin, such as cutting with a surgical knife or piercing the skin with a hypodermic needle. Intracutaneous agent delivery includes delivery via passive diffusion as well as delivery based upon external energy sources, such as electricity (e.g., iontophoresis) and ultrasound (e.g., phonophoresis).

The term “intracutaneous flux,” as used herein, means the rate of intracutaneous delivery of a drug.

The term “microprojection member” or “microneedle array,” and the like as used herein, generally connotes a microprojection grouping comprising a plurality of microprojections, preferably arranged in an array, for penetrating or piercing the stratum corneum. The microprojection member can be formed by etching or punching a plurality of microprojections from a thin sheet of metal or other rigid material, and folding or bending the microprojections out of the plane of the sheet to form a configuration. The microprojection member could alternatively be fabricated with other materials, including plastics or polymers, such as polyetheretherketone (PEEK). The microprojection member can be formed in other known techniques, such as injecting molding or micro-molding, microelectromechanical systems (MEMS), or by forming one or more strips having microprojections along an edge of each of the strip(s), as disclosed in U.S. Pat. Nos. 6,083,196; 6,091,975; 6,050,988; 6,855,131; 8,753,318; 9,387,315; 9,192,749; 7,963,935; 7,556,821; 9,295,714; 8,361,022; 8,633,159; 7,419,481; 7,131,960; 7,798,987; 7,097,631; 9,421,351; 6,953,589; 6,322,808; 6,083,196; 6,855,372; 7,435,299; 7,087,035; 7,184,826; 7,537,795; 8,663,155, and U.S. Pub. Nos. US20080039775; US20150038897; US20160074644; and US20020016562. As will be appreciated by one having ordinary skill in the art, when a microprojection array is employed, the dose of the therapeutic agent that is delivered can also be varied or manipulated by altering the microprojection array size, density, etc.

The term “microprojection” and “microneedles,” as used interchangeably herein, refers to piercing elements that are adapted to penetrate, pierce or cut into and/or through the stratum corneum into the underlying epidermis layer, or epidermis and dermis layers, of the skin of a living animal, particularly a mammal and, more particularly, a human. In one embodiment of the invention, the piercing elements have a projection length less than 1000 microns. In a further embodiment, the piercing elements have a projection length of less than 500 microns, more preferably less than 400 microns. The microprojections further have a width in the range of approximately 25 to 500 microns and a thickness in the range of approximately 10 to 100 microns. The microprojections may be formed in different shapes, such as needles, blades, pins, punches, and combinations thereof.

“Most bothersome other symptom” means a symptom, usually a migraine symptom, that is most bothersome to a patient, in addition to pain. Preferably, a most bothersome other symptom is identified by a patient at the start of a clinical trial. Usually, most bothersome other symptom is selected from nausea, photophonia, and phonophobia.

“Most bothersome other symptom freedom” means the patient reports an absence of the most bothersome other symptom at one or more pre-specified times after drug administration. Preferred times for migraine patients are 1 hour, 2 hours, and 4 hours. Preferred times for cluster headache patients are 15 minutes and 30 minutes.

“Nausea freedom” means the patient reports the absence of nausea at a pre-specific time period after drug administration. p The term “package” or “packaging” will be understood to also include reference to “storage” or “storing.”

“Pain freedom” means the patient reports an absence of headache pain (headache pain score=0) at one or more pre-specified time after drug administration. Preferred times for migraine patients are 1 hour, 2 hours, and 4 hours. Preferred times for cluster headache patients are 15 minutes and 30 minutes.

“Pain relief” means the patient reports a reduction in headache pain, a reduction from moderate or severe pain (headache pain score=3 or 2) to mild or no pain (headache pain score=1 or 0), at one or more pre-specified time period after drug administration. Preferred times for migraine patients are 1 hour, 2 hours, and 4 hours. Preferred times for cluster headache patients are 15 minutes and 30 minutes.

The terms “patient” and “subject” are used interchangeably herein and refer to a vertebrate, preferably a mammal. Mammals include, but are not limited to, humans.

The term “psilocybin” includes, without limitation, salts, enantiomers, racemic mixtures, polymorphs, analogs, prodrugs, derivatives, homologs and amorphous forms thereof

A drug “release rate,” as used herein, refers to the quantity of drug released from a dosage form or pharmaceutical composition per unit time, e.g., milligrams of drug released per hour (mg/hr). Drug release rates for drug dosage forms are typically measured as an in vitro rate of dissolution, i.e., a quantity of drug released from the dosage form or pharmaceutical composition per unit time measured under appropriate conditions and in a suitable fluid.

The term “stable” or “shelf-stable,” as used herein, refers to an agent formulation, means the agent formulation is not subject to undue chemical or physical change, including decomposition, breakdown, or inactivation. “Stable” as used herein, refers to a coating also means mechanically stable, i.e., not subject to undue displacement or loss from the surface upon which the coating is deposited.

The term “therapeutically effective” or “therapeutically effective amount,” as used herein, refer to the amount of the biologically active agent needed to stimulate or initiate the desired beneficial result. The amount of the biologically active agent employed in the coatings of the invention will be that amount necessary to deliver an amount of the biologically active agent needed to achieve the desired result. In practice, this will vary widely depending upon the particular biologically active agent being delivered, the site of delivery, and the dissolution and release kinetics for delivery of the biologically active agent into skin tissues.

The term “Tₘₐₓ” refers to the time from the start of delivery to the maximum plasma concentration of the biologically active agent.

The term “3,4-methylenedioxymethamphetamine” or “MDMA” includes, without limitation, salts, enantiomers, racemic mixtures, polymorphs, analogs, prodrugs, derivatives, homologs and amorphous forms thereof.

The term “lysergic acid diethylamide” or “LSD” includes, without limitation, salts, enantiomers, racemic mixtures, polymorphs, analogs, prodrugs, derivatives, homologs and amorphous forms thereof.

### B. INTRACUTANEOUS DELIVERY SYSTEM

The apparatus and method for intracutaneously delivering psilocybin, LSD or MDMA in accordance with this invention comprises an intracutaneous delivery system having a microneedle member (or system) having a plurality of microneedles (or array thereof) that are adapted to pierce through the stratum corneum into the underlying epidermis layer, or epidermis and dermis layers.

In one embodiment, the intracutaneous delivery system is a transdermal or intracutaneous drug delivery technology which comprises a disposable patch comprised of a microprojection member centered on an adhesive backing. The microprojection member comprises titanium (or other rigid material, including a plastic or polymeric material like polyetheretherketone (PEEK)) microneedles that are coated with a dry drug product formulation.

The patch is mounted in a retainer ring to form the patch assembly. The patch assembly is removably mounted in a handheld applicator to form the intracutaneous delivery system. The applicator ensures that the patch is applied with a defined application speed and energy to the site of intracutaneous administration. The applicator may be designed for single use or be reusable.

More particularly, the patch can comprise an array of about 3 to 6 cm² of titanium microneedles approximately 200-350 microns long, coated with a hydrophilic formulation of the relevant drug, and attached to an adhesive backing. The maximum amount of active drug that can be coated on a patch's microneedle array depends on the active moiety of the drug formulation, the weight of the excipients in the drug formulation, and the coatable surface area of the microneedle array. For example, patches with about 1 cm², 2 cm², 3 cm², 4 cm², 5 cm², and 6 cm² microneedle arrays may be employed. The patch is applied with a hand-held applicator that presses the microneedles into the skin to a substantially uniform depth in each application, close to the capillary bed, allowing for dissolution and absorption of the drug coating, yet short of the nerve endings in the skin. The typical patch wear time is about 15 to 45 minutes or less, decreasing the potential for skin irritation. Nominal applicator energies of about 0.20 to 0.60 joules are generally able to achieve a good balance between sensation on impact and array penetration. The actual kinetic energy at the moment of impact may be less than these nominal values due to incomplete extension of the applicator's spring, energy loss from breaking away the patch from its retainer ring, and other losses, which may comprise approximately total 25% of the nominal. Further description of Applicant's technology is described in U.S. Pat. Pub. No. US20190070103.

### 1. ARRAY DESIGN

A number of variables play a role in the type of array utilized for a particular active agent. For example, different shapes (e.g., shapes similar to an arrowhead as shown in FIG. 31, hook, conical, or the Washington monument, FIG. 1(A)-(B) in US20190070103) may enable higher drug loading capacity, while the length of the microprojections may be increased to provide more driving force for penetration. The stratum corneum has a thickness of about 10-40 μm, and microprojections must have an adequate size, thickness, and shape to penetrate and effect drug delivery through the stratum corneum. FIG. 31 in US20190070103, not drawn to scale, demonstrates how an array interacts with the skin, such that the micro-projections penetrate the stratum corneum and the substrate interfaces with the surface of the skin. It is advantageous to achieve a thicker coating on the microprojections, which will penetrate the stratum corneum, while avoiding applying coating to the substrate or the base (“streets”) of the array, which will not penetrate the stratum corneum. A larger surface area allows for a thicker coating without extending to the base or streets of the array. The coating is applied only to the microprojections. Further, the higher penetration force required for a more bulky projection with coating may be compensated by a longer length and lower density of projections per cm².

Exemplary intracutaneous delivery systems that may be used in the present disclosure include the drug delivery technologies described in U.S. Pat. Nos. 6,083,196; 6,091,975; 6,050,988; 6,855,131; 8,753,318; 9,387,315; 9,192,749; 7,963,935; 7,556,821; 9,295,714; 8,361,022; 8,633,159; 7,419,481; 7,131,960; 7,798,987; 7,097,631; 9,421,351; 6,953,589; 6,322,808; 6,083,196; 6,855,372; 7,435,299; 7,087,035; 7,184,826; 7,537,795; 8,663,155, and U.S. Pub. Nos. US20080039775; US20150038897; US20160074644; and US20020016562. The disclosed systems and apparatus employ piercing elements of various shapes and sizes to pierce the outermost layer (i.e., the stratum corneum) of the skin, and thus enhance the agent flux. The piercing elements generally extend perpendicularly from a thin, flat substrate member, such as a pad or sheet. The piercing elements are typically small, some having a microprojection length of only about 25 to 400 microns and a microprojection thickness of about 5 to 50 microns. These tiny piercing/cutting elements make correspondingly small microslits/microcuts in the stratum corneum for enhanced transdermal/intracutaneous agent delivery. The active agent to be delivered is associated with one or more of the microprojections, preferably by coating the microprojections with a psilocybin, LSD or MDMA-based formulation to form a solid, dry coating, or optionally, by the use of a reservoir that communicates with the stratum corneum after the microslits are formed, or by forming the microprojections from solid psilocybin, LSD or MDMA-based formulations that dissolve after application. The microprojections can be solid or can be hollow, and can further include device features adapted to receive and/or enhance the volume of the coating, such as apertures, grooves, surface irregularities or similar modifications, wherein the features provide increased surface area upon which a greater amount of coating can be deposited. The microneedles may be constructed out of stainless steel, titanium, nickel titanium alloys, or similar biocompatible materials, such as polymeric materials.

The present disclosure therefore encompasses patches and microneedle arrays having the following features:

Patch size: About 1 to 20 cm², or about 2 to 15 cm², or about 4 to 11 cm², or about 5 cm², or about 10 cm².

Substrate size: About 0.5 to 10 cm², or about 2 to 8 cm², or about 3 to 6 cm², or about 3 cm², or about 3.13 cm², or about 6 cm².

Array size: About 0.5 to 10 cm², or about 2 to 8 cm², or about 2.5 to 6 cm², or about 2.7 cm², or about 5.5 cm². or about 2.74 cm², or about 5.48 cm².

Density (microprojections/cm²): At least about 10 microprojections/cm², or in the range of about 200 to 2000 microprojections/cm², or about 500 to 1000 microprojections/cm², or about 650 to 800 microprojections/cm², or approximately 725 microprojections/cm².

Number of microprojections/array: About 100 to 4000, or about 1000 to 3000, or about or about 1500 to 2500, or about 1900 to 2100, or about 2000, or about 1987, or about 200 to 8000, or about 3000 to 5000, or about or about 3500 to 4500, or about 4900 to 4100, or about 4000, or about 3974.

Microprojection length: About 25 to 600 microns, or about 100 to 500 microns, or about 300 to 450 microns, or about 320 to 410 microns, or about 340 microns, or about 390 microns, or about 387 microns. In other embodiments, the length is less than 1000 microns, or less than 700 microns, or less than 500 microns. Accordingly, the microneedles penetrate the skin to about 25 to 1000 microns.

Tip length: About 100 to 250 microns, or about 130 to about 200 microns, or about 150 to 180 microns, or about 160 to 170 microns, or about 165 microns.

Microprojection width: About 10 to 500 microns, or about 50 to 300 microns, or about 75 to 200 microns, or about 90 to 160 microns, or about 250 to 400 microns, or about 300 microns, or about 100 microns, or about 110 microns, or about 120 microns, or about 130 microns, or about 140 microns, or about 150 microns.

Microprojection thickness: about 1 micron to about 500 microns, or about 5 microns to 300 microns, or about 10 microns to 100 microns, or about 10 microns to 50 microns, or about 20 microns to 30 microns, or about 25 microns.

Tip angle: about 10-70 degrees, or about 20-60 degrees or about 30 to 50 degrees, or about 35 to 45 degrees, or about 40 degrees.

Total active agent per array: About 0.1 mg to 10 mg, or about 0.5 mg to 5 mg, or about 1 mg to 4 mg, or about 1 mg, or about 1.9 mg, or about 3.8 mg, or about 2 mg per 3

Amount of inactive ingredient per array: About 0.1 to 10 mg, or about 0.2 to 4 mg, or about 0.3 mg to 2 mg, or about 0.6 mg, or about 0.63 mg, or about 1.3 mg, or about 1.26 mg. Alternatively, the amount of inactive ingredient is from one to three times less than the active agent, or from about 0.033 mg to about 3.33 mg.

Coating thickness: about 100 μm to about 500 μm, or about 200 μm to about 350 μm, or about 250 μm to about 290 μm, or about 270 μm.

Active agent per microprojection: About 0.01 to about 100 or about 0.1 to 10 or about 0.5 to 2 or about 1 μg, or about 0.96 μg.

In one embodiment of the invention, the microneedle member has a microneedle density of at least approximately 10 microprojections/cm², more preferably, in the range of at least approximately 200 to 750 microprojections/cm².

In one embodiment of the invention, the piercing elements (i.e., microprojections or microneedles) have a projection length less than 1000 microns. In a further embodiment, the piercing elements have a projection length of less than 700 microns. In other embodiments, the piercing elements have a projection length of less than 500 microns. Preferably, the microneedle length is between 300 and 400 microns in length. The microprojections further have a width in the range of about 100 to about 150 microns and a thickness in the range of about 10 to about 40 microns.

In one embodiment, the microprojection member is constructed out of stainless steel, titanium, nickel titanium alloys, or similar biocompatible materials, such as polymeric materials. The microprojection member includes a biocompatible coating that is disposed on the microneedles.

A particularly preferred embodiment has a patch area of about 5 cm² adhered to a titanium substrate with an area of about 3.1 cm² and a thickness of about 25 μm. The substrate is comprised of a microprojection array with an area of about 2.74 cm² containing about 1987 microprojections at a density of about 725 microprojections/cm². The dry formulation contained on each microprojection may have the approximate shape of an American football with a thickness that tapers down from a maximum of about 270 μm and comprises about 0.96 μg of psilocybin and about 0.32 of tartaric acid to about 1.9 mg of psilocybin and about 0.63 mg of tartaric acid per patch. Alternatively, the dry formulation may comprise MDMA or LSD in an amount of about 0.96 μg and about 0.32 of tartaric acid to about 1.9 mg of MDMA or LSD and about 0.63 mg of tartaric acid per patch.

FIG. 32 in US20190070103 demonstrates the shape of the microprojection, in a preferred embodiment, prior to bending (forming). Array forming is a process that bends the individual microprojections at right angles to the plane of the substrate. An array is placed over the forming tool, which contains cavities that are registered with the microprojections. An elastomeric forming disk is placed on top of the array and forced under pressure into the cavities in the forming tool. The elastomer flows into the cavities, causing the microprojections to be bent to the desired angle. The use of the elastomer has the advantage that no careful registration of the forming disk to the microprojections and the cavities is required in order to have effective array forming. As shown in FIG. 32 in US20190070103 the microprojections may be substantially rectangular, with a width of about 120±13 μm and a thickness of about 25.4±2.5 μm. The microprojections end with a triangular tip to facilitate penetration. The tip has an angle of 40±5 degrees, and is about 165±25 microns long. Prior to bending (forming) out from the substrate, the microprojections have a length of about 387±13 μm, and after bending, they protrude perpendicular to the substrate about 340 μm.

Another preferred embodiment has a patch area of about 5 cm² adhered to a titanium substrate of about 6 cm² to and a thickness of about 25 μm. The substrate is comprised of an array with an area of about 5.5 cm² containing about 4000 microprojections at a density of about 725 microprojections/cm². The dry formulation contained on each microprojection is in the approximate shape of an American football with a thickness that tapers down from a maximum of about 270 and comprises about 0.96 μ of psilocybin and about 0.32 of tartaric acid, or about 3.8 mg of psilocybin and about 1.3 mg of tartaric acid per patch. The microprojections have a length of about 387±13 μm, a width of about 120±13 μm, and a thickness of about 25.4±2.5 μm. The microprojections are rectangular, with a triangular tip to facilitate penetration. The tip has an angle of 40±5 degrees, and is about 165±25 microns long.

Alternatively, the dry formulation contained on each microprojection is in the approximate shape of an American football with a thickness that tapers down from a maximum of about 270 and comprises about 0.96 μ of LSD or MDMA and about 0.32 of tartaric acid, or about 3.8 mg of LSD or MDMA and about 1.3 mg of tartaric acid per patch.

The exact combination of bulk, length, and density that produces the desired penetration will vary, and may depend on the drug, its dose, the disease or condition to be treated and the frequency of administration. Thus, the drug delivery efficiency of a particular array (i.e., the amount of drug delivered to the bloodstream) will vary between about 40% to 100%, or about 40%, or about 50%, or about 60%, or about 70%, or about 80%, or about 90%, or about 100%.

### 2. IMPACT APPLICATOR

As illustrated in FIGS. 4(A)-(B), 5(A)-(E) in US20190070103, the intracutaneous drug delivery system of the present disclosure may further comprise an impact applicator having a body and a piston movable within the body, wherein the surface of the piston impacts the patch against the skin causing the microprojections to pierce the stratum corneum. The applicator is adapted to apply the microneedle array to the stratum corneum with an impact energy density of at least 0.05 joules per cm² in 10 milliseconds or less, or about 0.26 joules per cm² in 10 milliseconds or less, or about 0.52 joules per cm² in 10 milliseconds or less.

As illustrated in FIGS. 2(A) and 2(B) in US20190070103, the intracutaneous delivery system comprises a patch having an adhesive backing on one surface and a shiny metal surface on the other side comprised of the array of drug-coated microneedles. The patch may be applied to the skin by pressing the shiny metal surface against the skin either manually, or preferably by an applicator. Preferably, the applicator applies the patch to the skin with an impact energy density of 0.26 joules per cm² in 10 milliseconds or less. As shown in FIGS. 2A, 2B, 3A and 3B in US20190070103, the patch may be connected to and supported by a retainer ring structure forming a patch assembly. The retainer ring is adapted to fit onto the impact adaptor and removably attach the patch to the applicator. The retainer ring structure may comprise an inner ring and outer ring, which are designed to receive the adhesive patch and microneedle array. FIGS. 5(A)-(E) in US20190070103 demonstrate one embodiment of the claimed invention, in which the user facilitates the connection of the impact applicator to the retainer ring, which is already loaded with the patch and the microneedle array. As shown, once the retainer ring and impact applicator are connected, a user can unlock the impact applicator by twisting the applicator cap. FIG. 5(C) in US20190070103 shows that the user may then press the applicator downward on the skin to dispense the patch and apply it to the skin. The patch will removably attach to the patient's skin, and the retainer ring remains attached to the applicator. As shown in FIGS. 4(A) and 4(B) in US20190070103, the retainer ring reversibly attaches to the impact applicator such that the impact applicator can be reused during subsequent dosing events with additional patch assemblies and potentially for other active ingredients and disease states.

In another embodiment, the patch and applicator are supplied as a single, integrated unit, with packaging that ensures the stability and sterility of the formulation. The user removes the system from the packaging and applies the patch much as described above. The used applicator is then disposed of. This embodiment, while somewhat higher cost per dose, provides a system that is less complex, smaller, lighter, and easier to use.

The present disclosure can also be employed in conjunction with a wide variety of active transdermal systems (as opposed to passive, manual intracutaneous delivery devices described herein), as the disclosure is not limited in any way in this regard.

Some active transdermal systems utilize electrotransport. Illustrative electrotransport drug delivery systems are disclosed in U.S. Pat. Nos. 5,147,296; 5,080,646; 5,169,382 and 5,169,383, the disclosures of which are incorporated by reference herein in their entirety. One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane. In many instances, more than one of the noted processes may be occurring simultaneously to different extents. Accordingly, the term “electrotransport” is given herein its broadest reasonable interpretation, to include the electrically induced or enhanced transport of at least one charged or uncharged agent, or mixtures thereof, regardless of the specific mechanism(s) by which the agent is actually being transported with.

In addition, any other transport enhancing method, including but not limited to chemical penetration enhancement, laser ablation, heat, ultrasound, or piezoelectric devices, can be used in conjunction with the disclosure herein.

### 3. ACTIVE AGENTS AND BIOCOMPATIBLE COATING

The coating formulations applied to the microprojection member described above to form solid coatings are comprised of a liquid, preferably an aqueous formulation having at least one biologically active agent, which can be dissolved within a biocompatible carrier or suspended within the carrier. The biologically active agent may be psilocybin or MDMA, or pharmaceutically acceptable salts, analogs, enantiomers, racemic mixtures, derivatives, prodrugs, or polymorphs thereof, such as those described in U.S. Pat. No. 10,519,175.

Examples of pharmaceutically acceptable salts include, without limitation, acetate, propionate, butyrate, pentanoate, hexanoate, heptanoate, levulinate, chloride, bromide, citrate, succinate, maleate, glycolate, gluconate, glucoronate, 3-hydroxyisobutyrate, tricarballylate, malonate, adipate, citraconate, glutarate, itaconate, mesaconate, citramalate, dimethylolpropionate, tiglate, glycerate, methacrylate, i socrotonate, 13-hydroxibutyrate, crotonate, angelate, hydracrylate, ascorbate, aspartate, glutamate, 2-hydroxy-isobutyrate, lactate, malate, pyruvate, fumarate, tartrate, nitrate, phosphate, benzene sulfonate, methane sulfonate, sulfate and sulfonate.

The concentration of biologically active ingredient and excipients must be carefully controlled to achieve the desired amount of the active ingredient with an acceptable coating thickness, avoid wicking of the coating formulation onto the base of the microneedle array, maintain the uniformity of the coating, and ensure stability. In one embodiment, the active agent is present in the coating formulation at a concentration of between about 1% w/w to about 60% w/w, preferably between about 15% and 60%, or more preferably between 35% and 45%. The formulation may further comprise an acid at a concentration of between about 0.1% w/w to about 20% w/w. Such acid may be selected from tartaric acid, citric acid, succinic acid, malic acid, maleic acid, ascorbic acid, lactic acid, hydrochloric acid, either individually or in combination. In another embodiment, in the coating formulation, the active agent to acid ratio is about 1:1, about 2:1, about 3:1, about 4:1, or about 5:1.

The present disclosure further encompasses a coating formulation comprising about 33% w/w psilocybin, LSD or MDMA base and about 11% w/w tartaric acid. In some embodiments, the acid is one of tartaric acid, citric acid, succinic acid, malic acid or maleic acid, and is present in an amount of about 0.33% to 10% w/w, or about 8.33% to about 16.67% w/w, or about 13.33% w/w, or about 15% w/w, or about 6.67% w/w. In some embodiments, the coating formulation comprises 45% w/w of the active agent, 15% w/w of the acid, and 40% w/w of water.

Preferably, the psilocybin, LSD or MDMA is present in the coating formulation at a concentration comprised between 20% w/w and 50% w/w and a weak acid (tartaric acid, citric acid, malic acid, maleic acid) is present in the coating formulation between 6.67% w/w to 16.67% w/w.

Surfactants may be included in the coating formulation. Surfactants suitable for inclusion in the coating formulations include, but are not limited to, polysorbate 20 and polysorbate 80. Surfactants are commonly used to improve drug delivery as penetration enhancers. However, Applicant found that surfactants resulted in undulations in the coating formulation, which is indicative of an uneven film and is highly disadvantageous. Applicant found that the need for surfactants and other penetration enhancers can be avoided through the use of the claimed invention—specifically, through the claimed psilocybin, LSD or MDMA transdermal delivery patches. Furthermore, Applicant surprisingly found that microneedle coating avoided wicking, and the coating sufficiently adhered to the microprojections during the manufacturing process of the microneedle arrays, despite the lack of surfactant.

Antioxidants may be included in the coating formulation. Antioxidants suitable for inclusion in the coating formulations include, but are not limited to, methionine, ascorbic acid, and EDTA.

The coating formulation further comprises a liquid, preferably water, in an amount sufficient (qs ad) to bring the formulation to 100% prior to being dried onto the microneedles. The pH of the liquid coating formulation may be below about pH 8. In other cases, the pH is between about pH 3 and 7.4, or about or about pH 3 and 6.5, or about pH 3.5 to 4.5. Preferably, the pH of the coating formulation is below about pH 8. More preferably, the pH of the coating formulation is between 3 and 7.4. Even more preferably, the pH of the coating formulation is between 3.5 and 5.5.

The liquid coating formulations according to the present disclosure generally exhibit the ability to consistently coat the microneedles with adequate content and morphology, and result in a stable solid-state (dried) formulation, containing less than 5% water, preferably less than 3%. The liquid formulations are applied to the microneedle arrays and the microprojection tips thereof using an engineered coater which allows accurate control of the depth of the microprojection tips dipping into the liquid film. Examples of suitable coating techniques are described in U.S. Pat. No. 6,855,372, included herein by reference in its entirety. Accordingly, the viscosity of the liquid plays a role in microprojection member coating process as has been described. See Ameri, M.; Fan, S C.; Maa, Y F (2010); “Parathyroid hormone PTH(1-34) formulation that enables uniform coating on a novel transdermal microprojection delivery system;” Pharmaceutical Research, 27, pp. 303-313; see also Ameri M, Wang X, Maa Y F (2010); “Effect of irradiation on parathyroid hormone PTH(1-34) coated on a novel transdermal microprojection delivery system to produce a sterile product adhesive compatibility;” Journal of Pharmaceutical Sciences, 99, 2123-34.

The coating formulations comprising psilocybin, LSD or MDMA have a viscosity less than approximately 500 centipoise (cP) and greater than 3 cP, or less than approximately 400 cP and greater than 10 cP, or less than approximately 300 cP and greater than 50 cP, or less than 250 cP and greater than approximately 100 cP. In some embodiments, the viscosity of the liquid formulation prior to coating is at least 20 cP. In other embodiments, the viscosity is about 25 cP, or about 30 cP, or about 35 cP, or about 40 cP, or about 45 cP, or about 50 cP, or about 55 cP, or about 60 cP, or about 65 cP, or about 70 cP, or about 75 cP, or about 80 cP, or about 85 cP, or about 90 cP, or about 95 cP, or about 100 cP, or about 150 cP, or about 200 cP, or about 300 cP, or about 400 cP, or about 500 cP. In other embodiments, the viscosity is more than about 25 cP, or a more than about 30 cP, or more than about 35 cP, or more than about 40 cP, or more than about 45 cP, or more than about 50 cP, or more than about 55 cP, or more than about 60 cP, or more than about 65 cP, or more than about 70 cP, or more than about 75 cP, or more than about 80 cP, or more than about 85 cP, or more than about 90 cP, or more than about 95 cP, or more than about 100 cP, or more than about 150 cP, or more than about 200 cP, or more than about 300 cP, or more than about 400 cP, or less than about 500 cP. In a preferred embodiment, the viscosity of the coating formulation is more than about 80 cP and less than about 350 cP; in another preferred embodiment, the viscosity is more than about 100 cP and less than about 350 cP; and, in another preferred embodiment, the viscosity is more than about 100 cP and less than about 250 cP.

Once applied to the microprojections, the coating formulation may have an average thickness of about 10 to about 400 microns, or from about 30 to about 300 microns, or from about 100 microns to about 175 microns, or from about 115 to about 150 microns, or about 135 microns, as measured from the microprojection surface. Although it is preferable that the coating formulation have a uniform thickness covering the microprojection, the formulation may vary slightly as a result of the manufacturing process. As shown in FIG. 31 in US20190070103 the microprojections are generally coated uniformly because they penetrate the stratum corneum. In some embodiments, the microprojections are not coated the entire distance from the tip to the base; instead, the coating covers a portion of the length of the microprojection, measured from tip to the base, of at least about 10% to about 80%, or 20% to about 70%, or about 30% to about 60%, or about 40% to about 50% of the length of the microprojection.

The liquid coating formulation is applied to an array of microprojections so as to deliver a dose of the active agent in the amount of about 0.1 mg to 10 mg per array. In the case of psilocybin, LSD or MDMA, the dose is about 0.25 mg to about 10 mg, or about 1 mg or more, or about 1.9 mg or more, or about 2 mg or more, or about 3 mg or more, or about 3.8 mg or more, or about 4 mg or more, or about 5 mg or more delivered to the stratum corneum per array (via a patch or other form). In one embodiment, the amount of the psilocybin, LSD or MDMA contained in coating formulation is 1-1000 μg or 10-100 μg. In one embodiment, the array size is about 5.5 cm² comprising a dose of about 3.8 mg psilocybin, LSD or MDMA, or the array size is about 3 cm² comprising a dose of about 3.8 mg, or the array size is about 3 cm², comprising a dose of about 1.9 mg. The amount of psilocybin, LSD or MDMA or similar active agent per microprojection could range from about 0.001 to about 1000 or about 0.01 to about 100 μg, or about 0.1 to about 10 or about 0.5 to about 2 μg. In one embodiment, the amount of psilocybin, LSD or MDMA or similar active agent per microprojection is about 1 μg. The microprojection shape and size has a significant bearing on the drug loading capacity and on the effectiveness of drug delivery.

Importantly, the formulations of the present disclosure do not primarily rely on penetration enhancers to facilitate absorption of the active agent into the bloodstream. Penetration enhancers, such as Azone® and fatty acids, often cause skin irritation and have other disadvantages. Thus, the systems of the present disclosure are either completely free of a penetration enhancer, or are substantially free thereof In other embodiments, there is less than 15% w/w of penetration enhancer present, or less than 10% w/w, or less than 5% w/w, or less than 2.5% w/w, or less than 1% w/w present in the dried formulation.

The biologically active agent formulations are generally prepared as a solid coating by drying a coating formulation on the microprojection, as described in U.S. Application Pub. No. 2002/0128599. The coating formulation is usually an aqueous formulation. During a drying process, all volatiles, including water are mostly removed; however, the final solid coating may still contain about 1% w/w water, or about 2% w/w water, or about 3% w/w water, or about 4% w/w water, or about 5% w/w water. The oxygen and/or water content present in the formulations are reduced by the use of a dry inert atmosphere and/or a partial vacuum. In a solid coating on a microprojection array, the drug may be present in an amount of less than about 10 mg per unit dose or less than about 4 mg or less than about 3 mg or less than about 2 mg or less than about 1 mg. With the addition of excipients, the total mass of solid coating may be less than about 15 mg per unit dose.

The microprotrusion member is usually present on an adhesive backing, which is attached to a disposable polymeric retainer ring. This assembly is packaged individually in a pouch or a polymeric housing. In addition to the assembly, this package contains a dead volume that represents a volume of at least 3 mL. This large volume (as compared to that of the coating) acts as a partial sink for water. For example, at 20° C., the amount of water present in a 3 mL atmosphere as a result of its vapor pressure would be about 0.05 mg at saturation, which is typically the amount of residual water that is present in the solid coating after drying. Therefore, storage in a dry inert atmosphere and/or a partial vacuum will further reduce the water content of the coating resulting in improved stability.

According to the disclosure, the coating can be applied to the microprojections by a variety of known methods. For example, the coating may be only applied to those portions of the microprojection member or microprojections that pierce the skin (e.g., tips). The coating is then dried to form a solid coating. One such coating method comprises dip-coating. Dip-coating can be described as a method to coat the microprojections by partially or totally immersing the microprojections into a coating solution. By use of a partial immersion technique, it is possible to limit the coating to only the tips of the microprojections.

A further coating method comprises roller coating, which employs a roller coating mechanism that similarly limits the coating to the tips of the microprojections. The roller coating method is disclosed in U.S. Application Pub. No. 2002/0132054. As discussed in detail therein, the disclosed roller coating method provides a smooth coating that is not easily dislodged from the microprojections during skin piercing.

A further coating method that can be employed within the scope of the present invention comprises spray coating. Spray coating can encompass formation of an aerosol suspension of the coating composition. In one embodiment, an aerosol suspension having a droplet size of about 10 to 200 picoliters is sprayed onto the microprojections and then dried.

Pattern coating can also be employed to coat the microprojections. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

In one embodiment of the disclosure, the thickness of the dried coating formulations comprising psilocybin, LSD or MDMA range from about 10 to 100 microns as measured from the microprojection surface, or from about 20 to 80 microns, or from about 30 to 60 microns, or from about 40 to 50 microns. The desired coating thickness is dependent upon several factors, including the required dose and, hence, coating thickness necessary to deliver the dose, the density of the microprojections per unit area of the sheet, the viscosity, the solubility and concentration of the coating composition and the coating method chosen. The thickness of coating applied to microprojections can also be adapted to optimize stability of the psilocybin, LSD or MDMA. Known formulation adjuvants can also be added to the coating formulations provided they do not adversely affect the necessary solubility and viscosity characteristics of the coating formulation nor the physical integrity of the dried coating.

The coating is applied to the microneedles, which protrude from the base, or streets, of the microneedle array. The coating is applied to the tips of the microneedles, and is not intended to cover the microneedles and the surface of the microneedle array. This reduces the amount of drug per transdermal patch, which is advantageous in light of FDA Guidance on the danger of residual drug on transdermal delivery systems, which suggests that the amount of residual drug in a system should be minimized. See FDA Guidance for Industry, Residual Drug in Transdermal and Related Drug Delivery Systems (August 2011). Applicant's strategy was to maximize drug release into skin per unit area, without using an excess of drug for coating.

After a coating has been applied, the coating formulation is dried onto the microprojections by various means. The coated microprojection member may be dried in ambient room conditions. However, various temperatures and humidity levels can be used to dry the coating formulation onto the microprojections. Additionally, the coated member can be heated, stored under vacuum or over desiccant, lyophilized, freeze dried or similar techniques used to remove the residual water from the coating.

Coating may be conducted at ambient temperature utilizing a roller drum, rotating at 50 rpm, in a drug formulation reservoir (2 mL in volume) to produce a film of controlled thickness of around 270 μm in thickness. Further information about the coating process can be found in U.S. Pat. No. 6,855,372. Microprojection arrays are dipped into the drug film, and the amount of coating is controlled by the number of dips (passes) through the drug film.

During the drying process, there may be issues related to forming a uniform coating the microprojection with a controlled and consistent thickness. One common issue in transdermal patch coating, called “dripping” or “teardrop” formations, occurs when the coating is drying and the coating accumulates at the end of the microprojections in a “teardrop” shape. This teardrop shape can blunt the sharp end of the microneedle, potentially impacting the effectiveness and uniformity of penetration. Uneven layers of formulation on the microprojections results in uneven, and sometimes inadequate drug delivery. Additionally, the issues in the drying process cause issues of quality control in formulation coating. Preferred liquid coating formulations comprise psilocybin, LSD or MDMA in an amount of 30% w/w to about 60% w/w, preferably about 40% w/w to about 50% w/w, more preferably about 45% w/w, and tartaric acid in an amount of about 5% w/w to about 25% w/w, preferably about 10% w/w to about 20% w/w, more preferably about 15% w/w, in a liquid carrier, preferably water, more preferably deionized water. With these liquid coating formulations, Applicant surprisingly found that maintaining a viscosity of about 150 cP to about 350 cP, preferably about 200 cP to about 300 cP, more preferably about 250 centipoise, and a surface tension of about 50 mNm⁻¹ to about 72 mNm⁻¹, preferably about 55 mNm⁻¹ to about 65 mNm⁻¹, more preferably about 62.5 mNm⁻¹ was required to avoid dripping. Teardrop formation could be avoided while simultaneously allowing each dip of microprojections into the liquid coating formulation to pick up sufficient volume of liquid coating formulation, thereby achieving the desired drug dose with a minimum number of dips. When the viscosity and surface tension of the coating solution are high enough, the coated liquid does not quickly drip back or form a teardrop shape after dipping and before drying.

The products and methods described herein with respect to delivery of psilocybin, LSD or MDMA in a method of rapidly achieving therapeutic concentrations of psilocybin, LSD or MDMA for treatment of a variety of health conditions also extends to bioequivalent forms thereof

### 4. PACKAGING, STERILIZATION

Improved physical stability of the dry coated formulations provides not only the benefit of an increased storage or shelf life for the therapeutic agent itself, but enhances efficacy in that once stabilized in accordance with the compositions of and methods for formulating and delivering of the present invention, the therapeutic agents become useful in a greater range of possible formulations, and with a greater variety of therapeutic agent delivery means.

The present disclosure comprises an active agent formulation wherein the deterioration by oxygen and/or water is minimized and/or controlled by the manufacture and/or packaging of the active agent formulation in a dry inert atmosphere. The formulation may be contained in a dry inert atmosphere in the presence of a desiccant, optionally in a chamber or package comprising a foil layer.

The desiccant can be any known to those skilled in the art. Some common desiccants include, but are not limited to molecular sieve, calcium oxide, clay desiccant, calcium sulfate, and silica gel. The desiccant may be one that can be placed with the biologically active agent-containing formulation in the presence of an inert atmosphere in a package comprising a foil layer.

In another aspect, the active agent formulation is packaged in a chamber comprising a foil layer after the formulation is coated onto the microprojection array delivery device. In this embodiment, a desiccant is contained in the chamber, preferably attached to a chamber lid which comprises a foil layer, and the chamber is purged with dry nitrogen or other inert gas such as a noble gas prior to the delivery device-containing foil chamber being sealed by the foil lid. Any suitable inert gas can be used herein to create the dry inert atmosphere.

In one embodiment, the compositions of and methods for formulating and delivering psilocybin, LSD or MDMA suitable for intracutaneous delivery utilize a patch assembly. This patch assembly is manufactured and/or packaged in a dry inert atmosphere, and in the presence of a desiccant. In one embodiment, the patch assembly is manufactured in a dry inert atmosphere and/or packaged in a chamber comprising a foil layer and having a dry inert atmosphere and a desiccant. In one embodiment, the patch assembly is manufactured and/or packaged in a partial vacuum. In one embodiment, the patch assembly is manufactured and/or packaged in a dry inert atmosphere, and a partial vacuum. In one embodiment, patch assembly is manufactured in a dry inert atmosphere under a partial vacuum and/or packaged in a chamber comprising a foil layer and having a dry inert atmosphere, a partial vacuum, and a desiccant.

Generally, in the noted embodiments of the present invention, the inert atmosphere should have essentially zero water content. For example, nitrogen gas of essentially zero water content (dry nitrogen gas) can be prepared by electrically controlled boiling of liquid nitrogen. Purge systems can be also used to reduce moisture or oxygen content. A range for a partial vacuum is from about 0.01 to about 0.3 atmospheres.

In one embodiment, the compositions of and methods for formulating and delivering psilocybin, LSD or MDMA suitable for intracutaneous delivery using a microneedle delivery device, is manufactured and/or packaged in a dry inert atmosphere, preferably nitrogen or argon, and in the presence of a desiccant or oxygen absorber.

In one embodiment, the compositions of and methods for formulating and delivering psilocybin, LSD or MDMA suitable for intracutaneous delivery using a microneedle delivery device is manufactured and/or packaged in a foil lined chamber having a dry inert atmosphere, preferably nitrogen, and a desiccant or oxygen absorber.

In one embodiment, the compositions of and methods for formulating and delivering psilocybin, LSD or MDMA suitable for intracutaneous delivery using a microneedle delivery device is manufactured and/or packaged in a partial vacuum.

In one embodiment, the compositions of and methods for formulating and delivering psilocybin, LSD or MDMA suitable for intracutaneous delivery using a microneedle delivery device is manufactured and/or packaged in a foil lined chamber having a dry inert atmosphere, preferably nitrogen, a partial vacuum, and a desiccant or oxygen absorber.

In an aspect of this embodiment, the psilocybin, LSD or MDMA further comprises a biocompatible carrier. In another embodiment, there is an intracutaneous delivery system, adapted to deliver psilocybin, LSD or MDMA, comprising: (a) a microprojection member including a plurality of microprojections that are adapted to pierce the stratum corneum of a patient; (b) a hydrogel formulation comprised of psilocybin, LSD or MDMA, wherein the hydrogel formulation is in communication with the microprojection member; and (c) packaging purged with an inert gas and adapted to control environmental conditions sealed around the microprojection member, wherein the sealed package has been exposed to radiation to sterilize the microprojection member.

In another embodiment, there is an intracutaneous delivery system, adapted to deliver psilocybin, LSD or MDMA, comprising: (a) a microprojection member including a plurality of microprojections that are adapted to pierce the stratum corneum of a patient; (b) a solid film disposed proximate the microprojection member, wherein the solid film is made by casting a liquid formulation comprising psilocybin, LSD or MDMA, a polymeric material, a plasticizing agent, a surfactant and a volatile solvent; and (c) packaging purged with an inert gas and adapted to control environmental conditions sealed around the microprojection member, wherein the sealed package has been exposed to radiation to sterilize the microprojection member.

The present disclosure is also to a method for terminally sterilizing a patch assembly adapted to deliver psilocybin, LSD or MDMA, comprising the steps of: (a) providing a microprojection member having a plurality of microprojections that are adapted to pierce the stratum corneum of a patient having a biocompatible coating comprising psilocybin, LSD or MDMA disposed on the microprojection member; and (b) exposing the microprojection member to radiation selected from the group consisting of gamma radiation and e-beam, wherein the radiation is sufficient to reach a desired sterility assurance level. Such sterility assurance level may be 10⁻⁶ or 10⁻⁵. The method may further comprise sealing the micro-projection member with a desiccant inside packaging purged with an inert gas and exposing the packaged microprojection member to radiation selected from the group consisting of gamma radiation and e-beam radiation, wherein the radiation is sufficient to reach a desired sterility assurance level.

In an aspect of this embodiment, the method further comprises the step of mounting a patch comprised of a microprojection member attached to an adhesive backing on a pre-dried retainer ring to form a patch assembly, and subsequently sealing the microprojection member inside the packaging. In an aspect of this embodiment, the system further comprises a desiccant sealed inside the packaging with the patch assembly, and/or the packaging is purged with nitrogen, and/or the packaging comprises a pouch comprised of a foil layer. Preferably, the foil layer comprises aluminum.

The step of exposing the microprojection member to radiation may occur at approximately −78.5 to 25° C., or the member may be exposed to radiation at ambient temperature.

The radiation may be in the range of approximately 5 to 50 kGy, or approximately 10 to 30 kGy, or approximately 15 to 25 kGy, or approximately 21 kGy, or approximately 7 kGy. In one aspect of this embodiment, the radiation is delivered to the microprojection member at a rate of at least approximately 3.0 kGy/hr.

In one embodiment psilocybin, LSD or MDMA coated microneedles are exposed to a dose of radiation in the range of approximately 7-30 kGy. More preferably in the range of 15-30 kGys to a sterility assurance level of 10⁻⁵ to 10⁻⁶. p As described herein, Applicant developed a psilocybin, LSD or MDMA formulation which, when coated on the microneedle members of the present disclosure, is stable and maintains its amorphous character for at least 6 months, or at least 9 months, or at least 12 months, or at least 18 months, or at least 24 months after being exposed to radiation as described above.

In one embodiment, the dried psilocybin, LSD or MDMA formulation on the microneedles retains for at least 6 months approximately 100% of initial purity, or approximately 99% of initial purity, or approximately 98% of initial purity, or approximately 97% of initial purity, or approximately 96% of initial purity, or approximately 95% of initial purity, or approximately 90% of initial purity. In other aspects, such purity is retained for at least 9 months, or at least 12 months, or at least 18 months, or at least 24 months after packaging. In a further embodiment, the psilocybin, LSD or MDMA coating on the microneedles retains its purity as described in this paragraph, and also substantially maintains its amorphous character for at least 6 months, or at least 9 months or at least 12 months, or at least 18 months, or at least 24 months after packaging.

In one embodiment, a method for manufacturing a patch assembly for an intracutaneous delivery device adapted to deliver a psilocybin, LSD or MDMA, comprises the steps of: providing a microneedle member having a plurality of microneedles that are adapted to penetrate or pierce the stratum corneum of a patient having a biocompatible coating disposed on the microneedle member, the coating being formed from a coating formulation having psilocybin, LSD or MDMA and tartaric acid, citric acid, malic acid or maleic acid disposed thereon; sealing the microneedle member with a desiccant inside packaging purged with nitrogen and adapted to control environmental conditions surrounding the microneedle and exposing the microneedle member to radiation selected from the group consisting of gamma radiation, e-beam and x-ray wherein the radiation is sufficient to reach a desired sterility assurance level.

In accordance with another embodiment of the invention, a method for delivering stable biologically active agent formulations comprises the following steps: (i) providing a microprojection member having a plurality of microprojections, (ii) providing a stabilized formulation of biologically active agent; (iii) forming a biocompatible coating formulation that includes the formulation of stabilized biologically active agent, (iv) coating the microprojection member with the biocompatible coating formulation to form a biocompatible coating; (v) stabilizing the biocompatible coating by drying; and (vi) applying the coated microprojection member to the skin of a subject.

Additionally, optimal stability and shelf life of the agent is attained by a biocompatible coating that is solid and substantially dry. However, the kinetics of the coating dis-solution and agent release can vary appreciably depending upon a number of factors. It will be appreciated that in addition to being storage stable, the biocompatible coating should permit desired release of the therapeutic agent.

Encompassed herein is a method for terminally sterilizing a transdermal device adapted to deliver psilocybin, LSD or MDMA, comprising the steps of: providing a microprojection member having a plurality of microprojections that are adapted to penetrate or pierce the stratum corneum of a patient having a biocompatible coating disposed on the microprojection member, the coating being formed from a coating formulation having at least psilocybin, LSD or MDMA disposed thereon; and exposing the microprojection member to radiation selected from the group consisting of gamma radiation and e-beam, wherein the radiation is sufficient to reach a desired sterility assurance level. A further aspect of this method comprises the further step of sealing the microprojection member inside packaging adapted to control environmental conditions surrounding the microprojection member. In one aspect the packaging comprises a foil pouch. A further aspect of this method, comprises the further step of sealing a desiccant inside the packaging. Further, the method comprises the step of mounting the microprojection member on a pre-dried retainer ring prior to sealing the microprojection member inside the packaging. A further aspect of this method comprises the step of purging the packaging with an inert gas prior to sealing the packaging. In one embodiment, the inert gas comprises nitrogen.

### C. IN VIVO PHARMACOKINETICS (PK)

The patches of the present disclosure produce peak plasma concentrations of psilocin (active metabolite of psilocybin) of about 5 to about 13 ng/mL with a Tmax of no more than 30 minutes and an AUC from about 1000 to about 2000 ng*min/mL.

The patches of the present disclosure produce peak plasma concentrations of MDMA of about 0.5 to about 1 μg/mL with a Tmax of no more than 30 minutes and an AUC ranging from about 4000 to about 7000 ng*h/mL.

The patches of the present disclosure produce peak plasma concentrations of LSD of about 0.4 to about 0.7 μg/mL with a Tmax of no more than 45 minutes and an AUC ranging from about 150 to about 250 ng*h/mL.

### D. METHODS OF TREATMENT

The drug-device combinations of the present invention can be used to treat a variety of diseases and conditions, including depression, including major depressive disorder, anxiety, grief, post-traumatic stress disorder, Alzheimer Disease, mild cognitive impairment, obsessive-compulsive disorder, anorexia nervosa, migraine headache, cluster headache, post-traumatic headache, alcohol dependence, nicotine dependence, opioid use disorder, cocaine-related disorders, stage IV melanoma, cancer, Parkinson Disease, psychosis, adolescent behavior, adolescent development, and altered waking states of consciousness.

In a preferred embodiment of the present invention, there is a method for treatment or alleviation of depression, anxiety, and post-traumatic stress disorder to an individual in need thereof, comprising administration of a therapeutically effective amount of a psilocybin, LSD or MDMA-based agent, wherein the absorption of the psilocybin, LSD or MDMA-based agent results in a plasma Cₘₐₓ of less than 13 ng/mL in the case of psilocin, or less than 0.7 μg/mL in the case of LSD, or less than 1 μg/mL in the case of MDMA.

Doses include about 0.2 mg to about 10 mg psilocybin, LSD or MDMA. The dose may also be 0.48 mg, 0.96 mg, 1.9 mg, and 3.8 mg psilocybin, LSD or MDMA. Doses also include a single patch administration of either 1.0 mg, 1.9 mg, or 3.8 mg, or two patches of 1.9 mg. These doses can be delivered utilizing the patch(es) described herein and can be applied to the skin of any part of the body. In a preferred embodiment, the psilocybin, LSD or MDMA dose(s) is delivered via the patch to the skin.

### A. Depression

In one embodiment, the psilocybin, LSD or MDMA-coated microneedle patch as disclosed herein mitigates or prevents depression, as measured by one or more of the following:

1. The Beck Depression Inventory (BDI) to screen for depression and to measure behavioral manifestations and severity of depression. The BDI can be used for ages 13 to 80. The inventory contains 21 self-report items which individuals complete using multiple choice response formats.

2. The Center for Epidemiologic Studies Depression Scale (CES-D) as a screener for depression in primary care settings. It includes 20 self-report items, scored on a 4-point scale, which measure major dimensions of depression experienced in the past week. The CES-D can be used for children as young as 6 and through older adulthood.

3. The EQ-5D, a standardized, non-disease specific instrument for describing and evaluating health-related quality of life. The instrument measures quality of life in five dimensions: mobility, self-care, usual activities, pain/discomfort and anxiety/depression.

4. The Hamilton Rating Scale for Depression, abbreviated HDRS, HRSD or HAM-D, measures depression in individuals before, during and after treatment. The scale is administered by a health care professionals and contains 21 items, but is scored based on the first 17 items, which are measured either on 5-point or 3-point scales.

5. The 10-item Montgomery-Asberg Depression Rating Scale (MADRS) measures severity of depression in individuals 18 years and older. Each item is rated on a 7-point scale. The scale is an adaptation of the Hamilton Depression Rating Scale and has a greater sensitivity to change over time. The Social Problem-Solving Inventory-Revised (SPSI-RTM) is a self-report measure of social problem-solving strengths and weaknesses in individuals 13 years old and older.

### B. Anxiety

In one embodiment, the psilocybin, LSD or MDMA-coated microneedle patch as disclosed herein achieves rapid blood plasma concentrations after application during an anxiety episode or panic attack. Such patch provides relief against anxiety symptoms for at least 45 minutes post administration. The treatment or prevention of anxiety resulting from the application of the patch may be measured by one or more of the following:

1. Generalized Anxiety Disorder questionnaire-IV—GADQ-IV

2. Generalized Anxiety Disorder 7—GAD-7

3. Hamilton Anxiety Rating Scale—HARS

4. Leibowitz Social Anxiety Scale—LSAS

5. Overall Anxiety Severity and Impairment Scale—OASIS

6. Hospital Anxiety and Depression Scale—HADS

7. Depression Anxiety Stress Scales—DASS-21

8. Patient Health Questionnaire 4—PHQ-4

9. Penn State Worry Questionnaire—PSWQ

10. Social Phobia Inventory—SPIN

### C. PTSD

In one embodiment, the psilocybin, LSD or MDMA-coated microneedle patch as disclosed herein achieves rapid blood plasma concentrations after application during an episode of PTSD. Such patch provides relief against PTSD symptoms for at least 45 minutes post administration.

The treatment or prevention of PTSD resulting from the application of the patch may be measured by one or more of the following:

1. Clinician-Administered PTSD Scale for DSM-5 (CAPS-5).

2. PTSD Symptom Scale Interview (PSS-I and PSS-I-5).

3. Structured Clinical Interview; PTSD Module (SCID PTSD Module).

4. Structured Interview for PTSD (SIP or SI-PTSD).

5. Treatment-Outcome Posttraumatic Stress Disorder Scale (TOP-8).

6. Davidson Trauma Scale (DTS).

7. Impact of Event Scale—Revised (IES-R).

8. Mississippi Scale for Combat-related PTSD (MISS or M-PTSD).

9. Modified PTSD Symptom Scale (MPSS-SR).

10. PTSD Checklist for DSM-5 (PCL-5).

11. PTSD Symptom Scale Self-Report Version (PSS-SR).

12. Short PTSD Rating Interview (SPRINT).

### D. Migraine and Cluster Headache

In one embodiment, the psilocybin, LSD or MDMA-coated microneedle patch as disclosed herein achieves rapid blood plasma concentrations after application during a migraine or cluster headache attack. Such patch provides pain freedom and freedom from bothersome migraine or cluster headache symptoms for at least 45 minutes post administration. A patient's most bothersome migraine symptom in addition to pain is usually selected from sensitivity to light, particularly bright lights (photophobia), sensitivity to sound, particularly loud sounds (phonophobia), and nausea, although migraines can also be accompanied by vomiting, sensitivity to smell, aura, vision changes, numbness, tingling, weakness, vertigo, feeling lightheaded or dizzy, puffy eyelid, difficulty concentrating, fatigue, diarrhea, constipation, mood changes, food cravings, hives, and/or fever. Common symptoms of cluster headache include excruciating pain, often on one side of the head and generally situated in or around one eye, but which may radiate to other areas of face, head, neck and shoulders, restlessness, excessive tear production and redness in the eye on the affected side, stuffy or runny nose, forehead or facial sweating, pale skin (pallor), facial flushing, swelling around the eye on the affected side, and/or drooping eyelid.

In certain embodiments, the methods of treatment of migraine or cluster headache as described herein result in improvement with respect to the following therapeutic endpoints: Migraine Pain freedom at 1 hour, 2 hours, or 4 hours after dosing; Cluster headache pain freedom at 15 or 30 minutes after dosing, most bothersome other migraine symptom freedom at 1 hour or 2 hours after dosing; freedom from a patient's previously identified most bothersome other cluster headache symptom at 15 or 30 minutes after dosing, migraine pain relief at 1 hour, 2 hours or 4 hours; Cluster headache pain relief at 15 or 30 minutes after dosing, pain relief at 30 minutes; photophobia freedom at 2 hours; phonophobia freedom at 2 hours; pain relief at 15 minutes; pain relief at 3 hours; pain relief at 4 hours; nausea freedom at 2 hours; pain freedom at 30 minutes; pain freedom at 24 hours; and pain freedom at 48 hours. Further, there is an improvement in terms of treated patients requiring rescue medication. Improvement as to pain, most bothersome other symptom, photophobia, phonophobia, nausea, and other bothersome symptoms, is assessed sequentially, in a fixed-sequential testing method.

Additionally, the psilocybin , LSD or MDMA-coated patch as disclosed herein, when administered to a population of patients, results in a statistically significant number of patients experiencing pain freedom for up to 4 hours after treatment, or up to 3 hours after treatment, or up to 2 hours after treatment. In other cases, a statistically significant number of such patients experience at least a 1-level improvement in the headache pain scale score, or at least a 2-level improvement in the headache pain scale score.

### EXAMPLES

### Example 1—Treatment or Alleviation of Depression

A 3 cm² microneedle array with 350 um length, 70 um width microneedles coated with LSD and tartaric acid for total solids content 1.0 mg is used. A patient with depression may benefit from application of the invention.

A patient with depression may be treated with the psilocybin, LSD or MDMA-coated microneedle patch as disclosed herein resulting in mitigation or prevention of depression.

### Example 2—Treatment or Alleviation of Anxiety

A 3 cm² microneedle array with 350 um length, 70 um width microneedles coated with MDMA and tartaric acid for total solids content 2.5 mg is used. MDMA stimulates release of monoamines (serotonin, dopamine, and norepinephrine), elevates levels of the neurohormone oxytocin, reduces amygdala and right insular activity in response to negative emotional stimuli, increases superior frontal cortex activity, and increases connectivity between the amygdala and hippocampus. The effects of MDMA may reduce anxiety in the face of emotionally challenging thoughts or memories and can increase self-compassion. A patient with anxiety may benefit from application of the invention. p A patient with anxiety may be treated with the psilocybin, LSD or MDMA-coated microneedle patch as disclosed herein to achieve rapid blood plasma concentrations after application during an anxiety episode or panic attack.

### Example 3—Treatment or Alleviation of Post-Traumatic Stress Disorder

A 3 cm² microneedle array with 350 um length, 70 um width microneedles coated with LSD and tartaric acid for total solids content 1.0 mg is used. A patient with post-traumatic stress disorder may benefit from application of the invention.

A patient with PTSD may be treated with the psilocybin, LSD or MDMA-coated microneedle patch as disclosed herein to achieve rapid blood plasma concentrations after application during an episode of PTSD.

### Example 4—Treatment or Alleviation of Migraine, or Cluster Headache

A 3 cm² microneedle array with 350 um length, 70 um width microneedles coated with MDMA and tartaric acid for total solids content 2.5 mg is used. A patient with migraine, or cluster headache may benefit from application of the invention.

A patient suffering from migraine or cluster headache may be treated with the psilocybin, LSD or MDMA-coated microneedle patch as disclosed herein to achieve rapid blood plasma concentrations after application during a migraine or cluster headache attack.

While the invention has been described in conjunction with specific embodiments thereof, it is to be understood that the foregoing description as well as the examples are intended to illustrate and not limit the scope of the invention. Other aspects, advantages and modifications within the scope of the invention will be apparent to those skilled in the art to which the invention pertains.

1. An intracutaneous delivery system comprising a plurality of microprojections that are adapted to penetrate or pierce the stratum corneum of a human patient, the microprojections having a coating thereon comprising a therapeutically effective amount of psilocybin, LSD or MDMA.

2. The system of claim 1, wherein at least 95% of the psilocybin, LSD or MDMA is released within about 20 minutes after application of the system to the stratum corneum of the human patient.

3. The system of claim 2 wherein at least 95% of the psilocybin, LSD or MDMA is released within about 5 minutes after application of the system to the stratum corneum of the human patient.

4. The system of claim 1, wherein the coating further comprises an excipient.

5. The system of claim 1, wherein the therapeutically effective amount of psilocybin, LSD or MDMA is about 1 mg to about 10 mg.

6. The system of claim 5, wherein the therapeutically effective amount of psilocybin, LSD or MDMA is about 2 mg to about 5 mg.

7. The system of claim 1, wherein the coating further comprises an acid.

8. The system of claim 7, wherein the acid is tartaric acid.

9. The system of claim 1, wherein the system is stable at room temperature for at least 6 months.

10. (canceled)

11. A method for treating depression in a human patient in need thereof, comprising the steps of:
(a) providing an intracutaneous delivery system comprising a plurality of microprojections that are adapted to penetrate or pierce the stratum corneum of a human patient, the microprojections having a coating thereon comprising a therapeutically effective amount of psilocybin, LSD or MDMA,
(b) applying the microprojections to a selected area of skin of the patient.

12. The method of claim 11, wherein at least 95% of the psilocybin, LSD or MDMA is released within about 20 minutes after application of the microprojections to the patient.

13. (canceled)

14. The method of claim 11, wherein the therapeutically effective amount of psilocybin, LSD or MDMA is about 1 mg to about 10 mg.

15. (canceled)

16. The method of claim 11, wherein the system is self-administered.

17. The method of claim 11, wherein when the system is administered to a population of patients, a statistically significant number of patients are successfully treated for depression as measured by a method or scale selected from the group consisting of the Beck Depression Inventory (BDI), the Center for Epidemiologic Studies Depression Scale (CES-D), the EQ-5D, the HRSD, the MADRS, and combinations thereof.

18. The method of claim 12, wherein the wear time is about 5 to 30 minutes.

19. The system of claim 1, wherein the coating is a solid coating.

20. The system of claim 1, wherein the coating covers about 10% to 80% of the length of each microprojection measured from the tip to the base.

21. The system of claim 1, wherein the system comprises a disposable patch assembly having a plurality of microprojections disposed in an array of about 3 cm² to about 6 cm², the array having a density of about 200 to about 2000 microprojections/cm².

22. The system of claim 1, wherein the microprojections are rectangular, with a triangular tip to facilitate penetration of the stratum corneum of a human.

23. The system of claim 1, wherein the microprojections have:
(i) a length of about 25 to about 600 μm;
(ii) a width of about 10 μm to about 500 μm;
(iii) a thickness of about 1 μm to about 500 μm; and
(iv) a tip angle of about 30 to about 70 degrees.
